(19) United States
(12) Patent Application Publication
(10) Pub. No.: US 2018/0118664 A1
(43) Pub. Date: May 3, 2018

(54) Process for the Preparation of Fluorinated Diazoalkanes

(71) Applicant: Rheinisch-Westfälische Technische Hochschule (RWTH) Aachen, Aachen (DE)

(72) Inventors: Rene Königs, Erkelenz (DE); Katharina Julia Hock, Aachen (DE); Lucas Mertens, Eschweiler (DE)

(21) Appl. No.: 15/795,994

(22) Filed: Oct. 27, 2017

(30) Foreign Application Priority Data
Oct. 28, 2016 (DE) 10 2016 120 688.1

(51) Int. Cl. C07C 249/00

(52) U.S. Cl. CPC C07C 249/00

(57) ABSTRACT

The disclosure relates to a process for preparing a fluorinated diazoalkane in which the process is a continuous process and a β,β-difluoroalkylamine is reacted with an organic nitrite in a reactor, and in which the β,β-difluoroalkylamine and the organic nitrite are initially charged in separate vessels, and also to the use of the process for preparing a fluoroalkyl-substituted compound.

The disclosure relates to a process for preparing fluorinated diazoalkanes.

The synthesis of functionalized fluorinated compounds, as of heterocycles for example, is often important for the pharmaceutical and agrochemical industry in particular, since fluorinated compounds frequently have sought-after properties such as lipophilicity and metabolic stability. One of the issues with the synthesis of fluorinated organic compounds, however, is the use of highly reactive and correspondingly hazardous fluorinating reagents. A further disadvantage with existing preparative routes is that these frequently are multi-step, with losses at every stage, for example due to necessary working-up and purifying steps, reducing the yield of the desired compound. Therefore it is often important in preparative chemistry that existing synthesis strategies for the incorporation of fluorine atoms in organic compounds should be improved and/or novel ones be found.

One economical way to directly synthesise fluoroalkyl-substituted pyrazoles is the [2+3] cycloaddition reaction of fluoroalkyl-substituted diazomethanes with olefins or alkynes. The correspondingly fluoroalkyl-substituted diazomethanes needed are known to be synthesisable in an acid-catalysed reaction by use of nitrite. However, because of the explosion hazard posed by the resultant diazo compounds, conventional, batch processes typically call for strict safety precautions such as use of specific glass equipment, making it difficult to synthesise major amounts. A continuous process for preparing fluoroalkyl-substituted diazomethanes and their subsequent use in the synthesis of pyrazoles and pyrazolines was disclosed in Chem. Eur. J. 2016, 22, 9542-9545 “Fluoroalkyl-Substituted Diazomethanes and Their Application in a General Synthesis of Pyrazoles and Pyrazolines”. However, even with this process, the amount of the fluoroalkyl-substituted diazomethane synthesised is limited to the milligram range.

So there is a need for further improvements in the synthesis of fluoroalkyl-substituted diazomethanes. One problem addressed by the present disclosure was therefore that of providing a process which overcomes at least one of the aforementioned disadvantages of the prior art. One problem addressed by the present disclosure was more particularly that of providing a process useful for an industrial production of fluoroalkyl-substituted diazomethanes.

According to one embodiment, the problem is solved by a process for preparing a fluorinated diazoalkane wherein the process is a continuous process and a β,β-difluoroalkylamine is reacted with an organic nitrite in a reactor, wherein the β,β-difluoroalkylamine and the organic nitrite are initially charged to separate vessels.

It was found that, surprisingly, the process of the disclosure allows the continuous production of fluorinated diazoalkanes in major quantities. To wit, fluorinated diazoalkane was prepared from commercially available β,β-difluoroalkylamines in a continuous manner and thus used in gram amounts for organic synthesis. The process of the disclosure is the first to allow the continuous preparation of fluorinated diazoalkanes to be scaled up to the large industrial scale and also a scalable form of reaction management. The continuous preparation of the fluorinated diazoalkanes further allows its direct use in a two-step reaction whereby the fluorinated diazoalkane does not accumulate and the safety of the synthesis is significantly improved. The attendant availability of fluoroalkyl-substituted compounds further allows commercialization. The resulting fluoroalkyl-substituted compounds, especially di- or trifluoroalkyl-substituted heterocycles such as pyrazoles or pyrazolines, have huge commercial potential in the field of pharmaceuticals and agrochemicals in particular.

The term “continuous process” is to be understood as meaning a process which, in contradistinction to a discontinuous, batch process, proceeds continuously, without interruptions. Continuous processes can be advantageous for the production or processing of major amounts in particular.

The term “fluorinated diazoalkane” is to be understood as meaning a compound which contains at least one fluorine substituent as well as the diazo group. Starting from a β,β-difluoroalkylamine reactant, the fluorinated diazoalkanes obtained contain at least two fluorine substituents in the 2,2-position. The fluorinated diazoalkanes obtainable may also be referred to as fluoroalkyl-substituted diazomethanes in line with the simplest diazo compound, i.e., diazomethane. The fluorinated diazoalkanes obtainable may be for example 2,2-difluorodiazoethane or 2,2,2-trifluorodiazoethane. 2,2-Difluorodiazoethane may alternatively also be referred to as difluoromethyldiazomethane or 2,2-difluoromethyldiazomethane. 2,2,2-Trifluorodiazoethane may alternatively also be referred to as trifluoromethyldiazomethane or 2,2,2-trifluoromethyldiazomethane.

The term “vessel” is to be understood as meaning any receptacle suitable for storing liquid materials in particular. The vessel may also be referred to as a stock reservoir. The vessel may be for example a flask, a bottle, a tank, a syringe or any other container useful in industry or the laboratory. According to an embodiment of the disclosure, the β,β-difluoroalkylamine and the organic nitrite are initially charged to separate vessels. The β,β-difluoroalkylamine is preferably reacted with the organic nitrite in the presence of an organic acid, particularly acetic acid. The β,β-difluoroalkylamine, the organic nitrite and the acetic acid may be initially charged to two or more separate vessels. In an embodiment, the β,β-difluoroalkylamine is initially charged to a first vessel and the organic nitrite and acetic acid are initially charged conjointly to a second vessel, or the organic nitrite is initially charged to a first vessel and the β,β-difluoroalkylamine and acetic acid are initially charged conjointly to a second vessel. Here it is particularly provided that the β,β-difluoroalkylamine and the organic nitrite are initially charged to separate vessels. The acetic acid catalyst may in this case be initially charged not only conjointly with the nitrite but also conjointly with the amine. It has been determined that the yield with both measures is distinctly higher than when amine and nitrite are initially charged conjointly. It is believed that the two measures serve to prevent premature consumption of the amine and hence a reduction in yield.

There are embodiments wherein the β,β-difluoroalkylamine, the organic nitrite and the acetic acid are mixed in the reactor or before entry into the reactor. Here the components may be mixed in a mixer. The term “mixer” is to be understood as meaning a device suitable for commixing the reactants. The mixer may be for example a T-piece of a tube connector or of a pipe connector. The mixer in an embodiment is a minimixer, especially a micromixer. Component parts of this type are known from the field of microfluidics and are commercially available. Preference is given to using particularly micromixers, of glass for example, which may have additional commixing channels. Preferred mixers include for example Little Things Factory, MR-Lab Series, Vici or Upchurch Scientific PEEK Mixing Tee.

A pump, for example a metering pump, may be used to supply the mixer or the reactor with the reactants out of the particular vessels. The pump may be for example a syringe pump or an HPLC pump. The delivery rate of the pump may range from 10 μL/min to 100 mL/min.

The term “reactor” is to be understood as meaning a device in which a chemical reaction takes place. The reactor in an embodiment is a minireactor, especially a microreactor. Preference is given to a flow-through reactor, for example a flow pipe or a tube, whereto and wherefrom reactants and products are particularly simple to feed and withdraw, respectively, in continuous operation. The reactor may be for example of polytetrafluoroethylene (PTFE), fluoroethylenepropylene (FEP), perfluoroalkoxypolymers (PFAs) or stainless steel or some other alloy typically used in high performance or medium pressure liquid chromatography (HPLC or MPLC). Appropriate reactors are commercially available. The reactor may also be formed by a pipe piece or by a tube piece. For example, the reactor may be formed by some tubing, for example connected to and/or downstream of a mixer. Depending on the physical design of the device, it is possible for the reactor and the mixer to be formed by two separate devices. However, the mixer need not be a device designed to be separate from the reactor. Thus, in embodiments, the reactor and the mixer may be formed by one device. For example, in embodiments, the reactor may be formed by the mixer or co-perform this function, or vice versa, as long as the components-β,β-difluoroalkylamine, organic nitrite and acetic acid—are initially mixed.

The reactor, in an embodiment, is connected to a back pressure valve. The latter is capable of preventing the egress or formation of gases in the reactor or microreactor. The back pressure valve may be operated at a pressure of 20 psi to 80 psi for example.

The outlet of the reactor or microreactor may be connected to a further reaction vessel, for example a flask or a further reactor especially microreactor. The outlet of the reactor or microreactor may be preferably connected to a second microreactor, preferably in combination with a second micromixer. As a result, the fluorinated diazoalkane obtained can be reacted in a further reaction with a dipolarophile to afford a fluoroalkyl-substituted compound desired as end product. The second reaction step may be preferably carried out in a continuous regime, but can also be run as a batch reaction.

The β,β-difluoroalkylamine and the organic nitrite may be preferably reacted at elevated temperature in the reactor. In embodiments, the reaction of the β,β-difluoroalkylamine with the organic nitrite in the reactor is carried out at a temperature in the range of ≥40° C. to ≤100° C., preferably in the range of ≥40° C. to ≤80° C., preferably at a temperature in the range of ≥55° C. to ≤75° C. The reaction does give a good yield at temperatures as low as 40° C., but an increased temperature can lead to a faster course of the reaction as well as to a higher yield of fluorinated diazoalkane. At a temperature of 100° C. or higher, the yield may go down again. In an embodiment, it is particularly in a temperature range of ≥55° C. to ≤75° C. that a good ratio of yield for fluorinated diazoalkane to unconsumed amine was ascertained. The reaction may be carried out at a temperature of 55° C. for example. The temperature in the mixer preferably corresponds to that of the reactor. Correspondingly, the temperature in the mixer may be in the range of ≥40° C. to ≤100° C., preferably in the range of ≥40° C. to ≤80° C., more preferably at a temperature in the range of ≥55° C. to ≤75° C.

It was determined that the temperature was a factor having bearing on the reaction as well as the solvent used and the ratio of β,β-difluoroalkylamine to organic nitrite. The reaction time and also the acetic acid amount-of-substance fraction were further parameters studied. In embodiments, the residence time of the components β,β-difluoroalkylamine, organic nitrite and acetic acid in the reactor is in the range of ≥30 seconds to ≤1 hour, preferably in the range of ≥1 min to ≤20 min and preferably in the range of ≥2 min to ≤10 min. Even a residence time or reaction duration of just 30 seconds can be sufficient for nearly complete reaction between the amine and the nitrite, so the residual amount of unconsumed amine is low. This may be advantageous for a subsequent further reaction step in particular, since any catalyst needed will not be poisoned by the amine. The reaction duration may be adapted according to the reaction temperature. Carried out in a continuous manner, the reaction even reached good conversion from just a residence time in the range from ≥2 min to ≤10 min.

The volume of the reactor may be in the range from 200 μL to 100 ml. The size of the reactor used and the planned duration for the reaction may be used to set the flow rate at which the amine, the nitrite and the acetic acid or their mixtures are supplied to the mixer from the particular vessels and/or flow through the reactor. The β,β-difluoroalkylamine, the organic nitrite and the acetic acid or their mixtures may for example be supplied to the mixer and/or reactor at a flow rate ranging from ≥10 μL/min to ≤100 mL/min, preferably from ≥10 μL/min to ≤1 mL/min and more preferably from ≥50 μL/min to ≤≤100 μL/min. The β,β-difluoroalkylamine, the organic nitrite and the acetic acid may further flow through the reactor at a rate in the range from ≥10 μL/min to ≤100 mL/min, preferably in the range from ≥10 μL/min to ≤1 mL/min and more preferably in the range from ≥50 μL/min to ≤100 μL/min.

It may be advantageous for the continuous preparation of fluorinated diazoalkanes for these to be carried in a fluid. β,β-Difluoroalkylamine, organic nitrite and acetic acid are preferably charged in a nonaqueous solvent. Suitable nonaqueous solvents may be preferably selected from dichloromethane, dichloroethane and chloroform. Particularly chloroform may be a preferred solvent. It has further been determined in an embodiment to be advantageous to use dried chloroform. The use of dried chloroform gave better yields in this embodiment.

The concentration of the β,β-difluoroalkylamine in the stock reservoir vessel may be in the range from ≥0.1 M to ≤2 M, preferably in the range from ≥0.2 M to ≤0.8 M. The concentration of the organic nitrite in the stock reservoir vessel may be in the range from ≥0.1 M to ≤2.4 M, preferably in the range from ≥0.2 M to ≤1 M. In an embodiment, it has turned out to be advantageous to operate within these concentration ranges, since lower yields were obtained at higher concentrations.

The organic nitrite is preferably an alkyl nitrite, in an embodiment, especially a C₁-C₈-alkyl nitrite. Butyl nitrite, isobutyl nitrite, pentyl nitrite, neopentyl nitrite or tert-butyl nitrite have preferable usefulness. tert-Butyl nitrite in particular may be a favourable, commercially available organic source of nitrite. The organic nitrite may be used, relative to the β,β-difluoroalkylamine, in stoichiometric amounts or in molar excess. In embodiments, the organic nitrite is used in a ≥0.5-fold to ≤2-fold, preferably a ≥1-fold to ≤1.5-fold and more preferably a ≥1-fold to ≤1.2-fold molar excess based on the β,β-difluoroalkylamine. A slight excess of the organic nitrite gave particularly good yields of fluorinated diazoalkane.

Acetic acid was found to be a suitable catalyst for the synthesis of fluorinated diazoalkanes. Catalytic amounts of acetic acid have preferable usefulness. In embodiments, the amount-of-substance fraction of acetic acid is in the range of ≥1 mol % to ≤50 mol %, preferably in the range of ≥5 mol % to ≤40 mol % and more preferably in the range of ≥5 mol % to ≤10 mol %, based on the amount of substance of the β,β-difluoroalkylamine. Even just a 5 mol % acetic acid amount-of-substance fraction based on the β,β-difluoroalkylamine amount of substance led to very good yields by use of chloroform as solvent at a reaction temperature of 55° C. Similarly, a 40 mol % acetic acid amount-of-substance fraction based on the β,β-difluoroalkylamine amount of substance gave an about 40% yield of the desired difluoromethyldiazomethane in the course of a 2 minutes' reaction time at 75° C.

The amine group of the β,β-difluoroalkylamine reacts with the organic nitrite to form a diazo group. The β,β-difluoroalkylamine is therefore the precursor to the desired fluorinated diazo compound, preferably at least difluorinated in the 2,2-position. In embodiments, the β,β-difluoroalkylamine has the following general formula (1)

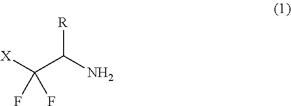

where:
R is selected from the group comprising H and/or substituted or unsubstituted alkyl, aryl, arylalkyl, cyclyl, heteroaryl or heterocyclyl,
X is selected from the group comprising H, F, Cl, CN, CO₂R′, CONR′, COR′, SO₂R′, SO₂NR′₂ and/or substituted or unsubstituted alkyl, aryl, arylalkyl, cyclyl, heteroaryl or heterocyclyl, and
R′ is selected, independently where applicable, from the group comprising H and/or substituted or unsubstituted alkyl, aryl, arylalkyl, cyclyl, heteroaryl or heterocyclyl.

For the purposes of the present disclosure, the term “heterocyclyl”, unless otherwise stated, is to be understood as meaning mono-, bi- or tricyclic heterocyclyl groups comprising one, two, three or four heteroatoms selected from the group comprising N, O and/or S. Monocyclic heterocyclyl groups are preferred heterocyclyl groups. Preferred heterocyclyl groups can comprise one or two heteroatoms selected from the group comprising N, O and/or S, preferably N. Preferred heterocyclyl groups are selected from the group comprising pyran, furan, piperidine and/or pyrrolidine.

For the purposes of the present disclosure, the term “heteroaryl”, unless otherwise stated, is to be understood as meaning mono-, bi- or tricyclic heteroaryl groups comprising one, two, three or four heteroatoms selected from the group comprising N, O and/or S. Monocyclic heteroaryl groups are preferred heteroaryl groups. Preferred monocyclic heteroaryl groups have one or two heteroatoms. Preferred heteroaryl groups are selected from the group comprising pyridine, pyrimidine, pyrrole, pyrazole, imidazole, thiazole, oxazole and/or isoxazole.

The term “C₁₋₈-alkyl”, unless otherwise stated, comprehends straight-chain or branched alkyl groups having 1 to 8 carbon atoms. The term “Me” refers to a methyl group and the term “Et” refers to an ethyl group. The term “C₅₋₆-heteroaryl”, unless otherwise stated, refers to an aromatic ring system having 5 or 6 carbon and heteroatom atoms.

For the purposes of the present disclosure, the term “arylalkyl”, unless otherwise stated, is to be understood as meaning a group which is attached via the latter moiety, in that for example an arylalkyl group is attached via the alkyl moiety. The number of carbon atoms is preferably 6 for the aryl group and from 1 to 3 for the alkyl moiety. Benzyl is a preferred arylalkyl group.

The alkyl, aryl, cyclyl, heteroaryl and heterocyclyl groups may be substituted, for example with one or more groups selected from the group comprising F, Cl, CF₃, CF₂H, CHF₂, O—C₁₋₈-alkyl, O—C₃₋₆-cycloalkyl, OCF₃, OCF₂H, SCF₃, SCF₂H, S—C₁₋₈-alkyl, S—C₃₋₆-cycloalkyl, SO—C₁₋₈-alkyl, SO—C₃₋₆-cycloalkyl, SO₂—C₁₋₈-alkyl, SO₂—C₃₋₆-cycloalkyl, CONH₂, CONHMe, CONMe₂ and/or CO₂—C₁₋₆-alkyl.

In embodiments of the β,β-difluoroalkylamine of general formula (1),
R is selected from the group comprising H and/or substituted or unsubstituted C₁₋₈-alkyl, phenyl, benzyl, C₃₋₆-cyclyl, C₅₋₆-heteroaryl or C₃₋₆-heterocyclyl,
X is selected from the group comprising H, F, Cl, CN, CO₂R′, CONR′, COR′, SO₂R′, SO₂NR′₂ and/or substituted or unsubstituted C₁₋₈-alkyl, phenyl, benzyl, C₃₋₆-cyclyl, C₅₋₆-heteroaryl or C₃₋₆-heterocyclyl, and
R′ is selected, independently where applicable, from the group comprising H and/or substituted or unsubstituted C₁₋₈-alkyl, phenyl, benzyl, C₃₋₆-cyclyl, C₅₋₆-heteroaryl or C₃₋₆-heterocyclyl.

In embodiments of the β,β-difluoroalkylamine of formula (1), X is hydrogen, fluorine or a perfluoro group selected from CF₃, C₂F₅ C₃F₇ and/or C₄F₉ or X is selected from the group comprising H, F, CF₃, CF₂H, CFH₂ and/or C₂ F₅. In further embodiments of the β,β-difluoroalkylamine of formula (1), X is ═H and R is ═H. In further embodiments of the β,β-difluoroalkylamine of formula (1), X is ═F and R is ═H. In likewise embodiments of the β,β-difluoroalkylamine of formula (1), X is ═F and R is selected from the group comprising methyl, ethyl, phenyl and/or phenyl substituted with Me, OMe, F, Cl, Br or COOC₁₋₆-alkyl, especially COOMe, COOEt, COOtert.butyl.

Preferred β,β-difluoroalkylamines are 2,2-difluoroethylamine and 2,2,2-trifluoroethylamine. Further preferred β,β-difluoroalkylamines are selected from the group comprising: 2,2,3,3,3-pentafluoropropylamine, 2,2,3,3,4,4,4-heptafluoro-butylamine, 2,2,3,3,4,4,5,5,5-nonafluoropentylamine, 1,1,1-trifluoropropan-2-amine, 1,1,1-trifluorobutan-2-amine, 1,1,1-trifluoro-4-phenylbutan-2-amine, 2,2,2-trifluoro-1-phenylethan-1-amine, 2,2,2-trifluoro-1-(4-fluorophenyl)ethan-1-amine and/or 2,2,2-trifluoro-1-(4-methylphenyl)ethan-1-amine.

In an embodiment, one advantage of the process is that the fluorinated diazoalkane obtained does not have to be extracted, purified or worked up before being further used in subsequent steps of synthesis. The fluorinated diazoalkane obtained is thus directly usable in a subsequent reaction to prepare a desired fluorinated organic compound. For this, the fluorinated diazoalkane obtained may be reacted with a dipolarophile to form the fluoroalkyl-substituted compound desired as end product. The second reaction step is preferably carried out in a continuous regime, but can also be run as a batch reaction.

One further aspect of the disclosure relates to the use of the process according to the disclosure for preparing a fluoroalkyl-substituted compound. The process is usable, for example, as part of a two-step reaction for preparation of cyclopropanes by a subsequent cyclopropanation or for preparation of pyrazoles and pyrazolines by subsequently reacting the fluorinated diazoalkane with a dipolarophile in a 1,3-dipolar cycloaddition. Preferred fluoroalkyl-substituted compounds are di- or trifluoroalkyl-substituted pyrazoles, pyrazolines and cyclopropanes.

One further aspect of the disclosure correspondingly relates to a process for preparing a fluoroalkyl-substituted compound, especially di- or trifluoroalkyl-substituted pyrazoles, pyrazolines or cyclopropanes, wherein the process comprises the steps of:
a) preparing a fluorinated diazoalkane as per the above-described process, and
b) reacting the fluorinated diazoalkane in a cyclopropanation reaction or with a dipolarophile in a 1,3-dipolar cycloaddition reaction

Regarding a description of the preparation of the fluorinated diazoalkane as per step a), the above description is incorporated by reference. The conversion of the fluorinated diazoalkane in a cyclopropanation reaction or with a dipolarophile in a 1,3-dipolar cycloaddition reaction as per step b) may be effected according to conditions known to the person skilled in the art. A preferred process is the preparation of di- or trifluoroalkyl-substituted pyrazoles and pyrazolines by a 1,3-dipolar cycloaddition reaction.

Step b) may be carried out as a conventional, discontinuous batch reaction. The fluorinated diazoalkane may for this be added to the dipolarophile in a flask for example. An advantage of a discontinuous batch reaction is that the diazoalkane does not have to be isolated and a rapid and standardised protocol is available for step b) for reactions on a small scale. Yields of more than one gram of difluoromethyl-substituted pyrazoline were even obtained in a discontinuous protocol for step b).

Alternatively, step b) may be carried out in a continuous manner. For this, the outlet of the first reactor used for step a) may be connected to a further reactor especially microreactor. The dimensioning of the second reactor is choosable irrespective of that of the first reactor and within the framework of the desired reaction regime. For a continuous reaction, a first microreactor may be used in step a) and a second microreactor in step b). An advantage of a continuous reaction is that increased safety can be established for preparing and handling diazomethanes. An advantage further resides in the scalability of a continuous form of production. It was found that the continuous as well as the discontinuous protocol in step b) led to very good yields of di- and trifluoroalkyl-substituted pyrazoles and pyrazolines.

The outlet of the first reactor or microreactor is preferably connected to a second mixer or micromixer, respectively. The dipolarophile may likewise be injected into the second mixer.

The diazoalkane and the dipolarophile, in both a continuous and a discontinuous protocol, may be reacted at a temperature in the range from ≥0° C. to ≤75° C. and/or for a reaction time in the range from ≥10 min to ≤14 h, optionally with stirring and/or adding a catalyst. The cyclopropanation reaction preferably takes place for a reaction time in the range from ≥10 min to ≤6 hours and/or at a temperature in the range from ≥0° C. to 40° C. in a batch reaction in the presence of a suitable catalyst.

The dipolarophile is preferably dissolved in a solvent. Suitable nonaqueous solvents are preferably selected from dichloromethane, dichloroethane and chloroform. The solvent may correspond to the solvent used in step a). Chloroform in particular is a preferred solvent for preparing di- or trifluoroalkyl-substituted cyclopropanes. Dichloromethane has proved to be a preferred solvent for preparing aryl- and alkylsulfonyl-substituted pyrazolines.

Preferred di- or trifluoroalkyl-substituted pyrazoles and pyrazolines are selected from the group comprising:
methyl 3-(difluoromethyl)-1H-pyrazole-5-carboxylate,
ethyl 3-(difluoromethyl)-1H-pyrazole-5-carboxylate, tert-butyl 3-(difluoromethyl)-1H-pyrazole-5-carboxylate,
5-benzyl-3-(difluoromethyl)-1,6a-dihydropyrrolo[3,4-c]pyrazole-4,6 (3aH, 5H)-dione,
1-(3-(difluoromethyl)-1H-pyrazol-5-yl)ethanone,
dimethyl 3-(difluoromethyl)-1H-pyrazole-4,5-dicarboxylate,
tert-butyl 3-(difluoromethyl)-4,5-dihydro-1H-pyrazole-5-carboxylate,
1-(3-(difluoromethyl)-4,5-dihydro-1H-pyrazol-5-yl)ethanone,
tert-butyl 3-(trifluoromethyl)-1H-pyrazole-5-carboxylate,
methyl 3-(trifluoromethyl)-1H-pyrazole-5-carboxylate,
ethyl 3-(trifluoromethyl)-1H-pyrazole-5-carboxylate, 1-(3-(trifluoromethyl)-1H-pyrazol-5-yl)ethanone,
dimethyl 3-(trifluoromethyl)-1H-pyrazole-4,5-dicarboxylate,
1-(3-(trifluoromethyl)-4,5-dihydro-1H-pyrazol-5-yl)ethanone,
tert-butyl 3-(trifluoromethyl)-4,5-dihydro-1H-pyrazole-5-carboxylate,
ethyl 5-(difluoromethyl)-4-(trifluoromethyl)-4,5-dihydro-1H-pyrazole-3-carboxylate and/or
ethyl 3-(difluoromethyl)-4-(trifluoromethyl)-1H-pyrazole-5-carboxylate.

Preferred di- or trifluoroalkyl-substituted pyrazoles and pyrazolines are selected from the group comprising methyl 3-(difluoromethyl)-1H-pyrazole-5-carboxylate, ethyl 3-(difluoromethyl)-1H-pyrazole-5-carboxylate and/or tert-butyl 3-(difluoromethyl)-1H-pyrazole-5-carboxylate. An access to difluoromethylcarboxyl-substituted pyrazoles is provided in an advantageous manner. The process may advantageously represent an economical and scalable method to directly synthesise fluoroalkyl-substituted pyrazoles and pyrazolines from propiolic esters as dipolarophile.

Preferred di- or trifluoroalkylsulfonyl-substituted pyrazoles and pyrazolines are selected from the group comprising:
3-(methylsulfonyl)-5-(trifluoromethyl)-4,5-dihydro-1H-pyrazole,
3-(ethylsulfonyl)-5-(trifluoromethyl)-4,5-dihydro-1H-pyrazole,
3-(benzylsulfonyl)-5-(trifluoromethyl)-4,5-dihydro-1H-pyrazole,
3-(phenylsulfonyl)-5-(trifluoromethyl)-4,5-dihydro-1H-pyrazole,
3-((4-methylphenyl)sulfonyl)-5-(trifluoromethyl)-4,5-dihydro-1H-pyrazole,
3-((4-fluorophenyl)sulfonyl)-5-(trifluoromethyl)-4,5-dihydro-1H-pyrazole,
3-((4-chlorophenyl)sulfonyl)-5-(trifluoromethyl)-4,5-dihydro-1H-pyrazole,
3-((4-bromophenyl)sulfonyl)-5-(trifluoromethyl)-4,5-dihydro-1H-pyrazole,
3-((4-methoxyphenyl)sulfonyl)-5-(trifluoromethyl)-4,5-dihydro-1H-pyrazole,
3-((3-methylphenyl)sulfonyl)-5-(trifluoromethyl)-4,5-dihydro-1H-pyrazole,
3-((2-methylphenyl)sulfonyl)-5-(trifluoromethyl)-4,5-dihydro-1H-pyrazole,
3-(methylsulfonyl)-5-(difluoromethyl)-4,5-dihydro-1H-pyrazole,
3-(ethylsulfonyl)-5-(difluoromethyl)-4,5-dihydro-1H-pyrazole,
3-(benzylsulfonyl)-5-(difluoromethyl)-4,5-dihydro-1H-pyrazole,
3-(phenylsulfonyl)-5-(difluoromethyl)-4,5-dihydro-1H-pyrazole,
3-((4-methylphenyl)sulfonyl)-5-(difluoromethyl)-4,5-dihydro-1H-pyrazole,
3-((4-fluorophenyl)sulfonyl)-5-(difluoromethyl)-4,5-dihydro-1H-pyrazole,
3-((4-chlorophenyl)sulfonyl)-5-(difluoromethyl)-4,5-dihydro-1H-pyrazole,
3-((4-bromophenyl)sulfonyl)-5-(difluoromethyl)-4,5-dihydro-1H-pyrazole,
3-((4-methoxyphenyl)sulfonyl)-5-(difluoromethyl)-4,5-dihydro-1H-pyrazole,
3-((3-methylphenyl)sulfonyl)-5-(difluoromethyl)-4,5-dihydro-1H-pyrazole,
3-((2-methylphenyl)sulfonyl)-5-(difluoromethyl)-4,5-dihydro-1H-pyrazole,
3-(methylsulfonyl)-5-(methyl)-5-(trifluoromethyl)-4,5-dihydro-1H-pyrazole,
3-(phenylsulfonyl)-5-(methyl)-5-(trifluoromethyl)-4,5-dihydro-1H-pyrazole,
3-(methylsulfonyl)-5-phenethyl-5-(trifluoromethyl)-4,5-dihydro-1H-pyrazole,
3-(methylsulfonyl)-5-(phenyl)-5-(trifluoromethyl)-4,5-dihydro-1H-pyrazole,
3-(methylsulfonyl)-5-(4-Methylphenyl)-5-(trifluoromethyl)-4,5-dihydro-1H-pyrazole and/or
3-(methylsulfonyl)-5-(4-Fluorophenyl)-5-(trifluoromethyl)-4,5-dihydro-1H-pyrazole.

Such fluoroalkyl-substituted, and especially sulfonyl- and fluoroalkyl-substituted, pyrazoles and pyrazolines may be important for the pharmaceutical and agrochemical industry because they may exhibit elevated lipophilicity and metabolic stability. The process may advantageously also represent an economical and scalable method to directly synthesise fluoroalkyl-substituted and also sulfonyl- and fluoroalkyl-substituted pyrazoles and pyrazolines. It was found that a wide spectrum of various aromatic compounds and aliphatic vinyl sulfones are convertible into the corresponding trifluoromethyl- and sulfonyl-substituted pyrazolines. These were further obtained in outstanding yield.

In embodiments, the dipolarophile has the following general formula (2) or (3):

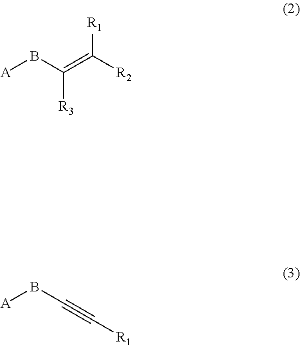

where:
A is selected from the group comprising substituted or unsubstituted alkyl, aryl, arylalkyl, cyclyl, heteroaryl, heterocyclyl, alkoxy, aryloxy, NR″₂ and/or NHR″,
B is selected from the group comprising CO and/or SO₂,
R₁, R₂ and R₃ are each independently selected from the group comprising H, F, Cl, C₁₋₃-alkyl, CF₃, CF₂H, CFH₂, phenyl, COR″ and/or SO₂R′″,
R″ is selected, independently where applicable, from the group comprising C₁₋₈-alkyl, phenyl and/or C₅₋₆-heteroaryl, and
R′″ is selected from the group comprising H and/or substituted or unsubstituted alkyl, aryl, arylalkyl, cyclyl, heteroaryl or heterocyclyl,
where in the case of the dipolarophile of formula (2) A and R₁ are capable of conjointly forming a 5-, 6- or 7-membered ring which is present as ketone, lactone, lactam, sulfone, sulfonic ester, sultam, anhydride or imide.

In further embodiments of the dipolarophile of general formulae (2) and (3),
A is selected from the group comprising C₁₋₈-alkyl, phenyl, benzyl, C₃₋₆-cyclyl, C₆-heteroaryl, C₃₋₆-heterocyclyl, C₁₋₈-alkoxy, phenyloxy, NR″₂ and/or NHR″;
B is selected from the group comprising CO and/or SO₂,
R₁, R₂ and R₃ are each independently selected from the group comprising H, F, Cl, C₁₋₃-alkyl, CF₃, CF₂H, CFH₂, phenyl, COR′, and/or SO₂R′,
R″ is selected, independently where applicable, from the group comprising C₁₋₈-alkyl, phenyl, benzyl and/or C₅₋₆-heteroaryl, and
R′″ is selected from the group comprising H and/or substituted or unsubstituted C₁₋₈-alkyl, phenyl, benzyl, C₃₋₆-cyclyl, C₅₋₆-heteroaryl or C₃₋₆-heterocyclyl,
where in the case of the dipolarophile of formula (2) A and R₁ are capable of conjointly forming a 5-, 6- or 7-membered ring which is present as ketone, lactone, lactam, sulfone, sulfonic ester, sultam, anhydride or imide.

Preferred dipolarophiles are selected from the group comprising methyl propiolate, ethyl propiolate, tert-butyl propiolate, methyl acrylate, ethyl acrylate and/or tert-butyl acrylate.

It may be a further advantage that the process is suitable for preparing fluorinated cyclopropanes. In these embodiments, the dipolarophile is preferably selected from unsubstituted styrene or substituted styrene such as indene. Styrene has the IUPAC name of phenylethene. Styrene may for example be substituted with substituents selected from F, Cl, Br, C₁₋₈-alkyl, especially methyl or tert-butyl. The process of the disclosure thus provides the first efficient, simple and economical synthesis of difluoromethyl-substituted cyclopropanes from commercially available olefins and amines. Difluoroethylamine is the preferred β,β-difluoroalkylamine for preparing di- or trifluoroalkyl-substituted cyclopropanes.

A process is altogether provided for using commercially available reactants to prepare fluoroalkyl-substituted diazomethanes under industrially scalable conditions in good yield and increased safety. The process allows the upscaling of this reaction to the large industrial scale. The continuous availability of the fluoroalkyl-substituted diazomethanes further permits a two-step protocol for the synthesis of fluoroalkyl-substituted, and especially di- and trifluoroalkyl-substituted or sulfonyl- and fluoroalkyl-substituted, pyrazoles and pyrazolines in high yields. These compounds are therefore available for commercialisation and further development.

Examples and figures follow to illustrate the present disclosure.

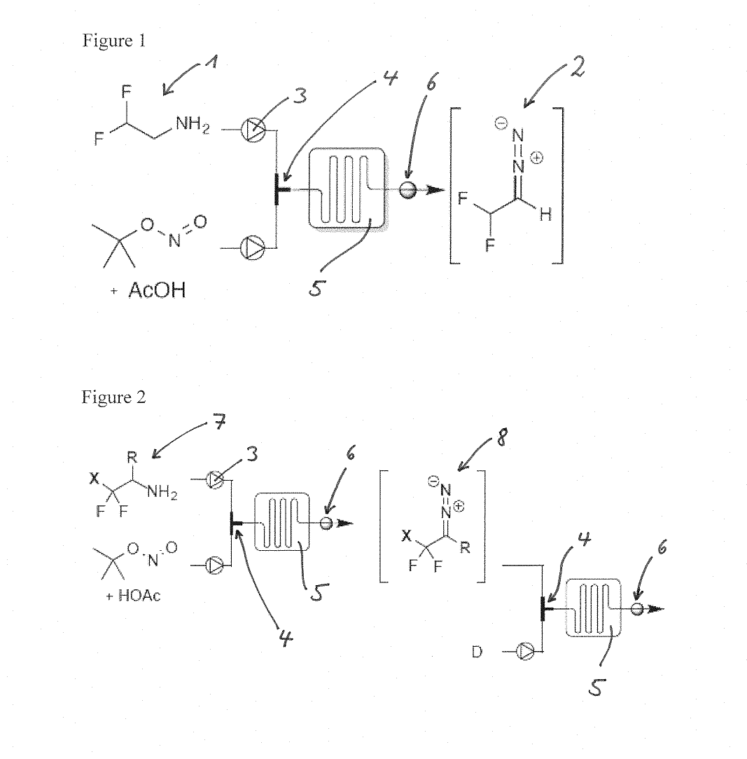

FIG. 1 shows the preparation of a fluorinated diazoalkane, exemplified by difluoroethylamine, as per an embodiment of the process according to the disclosure.

FIG. 2 shows the preparation of a di- or trifluoroalkyl-substituted compound by preparing a fluorinated diazoalkane and subsequently reacting the fluorinated diazoalkane with a dipolarophile.

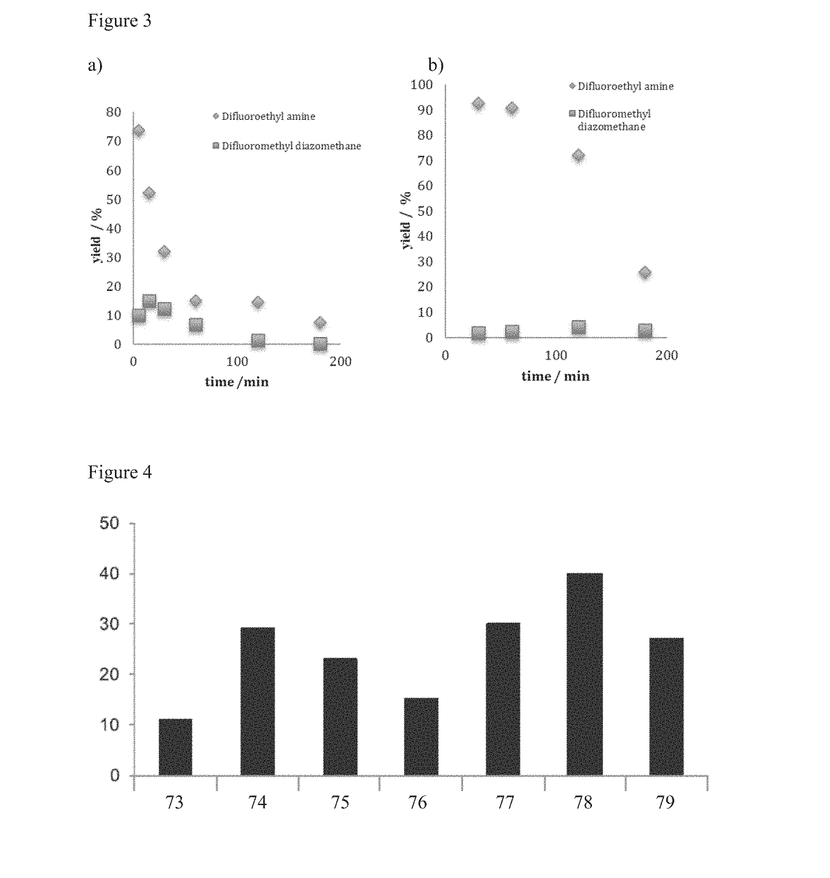

FIG. 3 shows the yields for the preparation of difluoromethyldiazomethane as per Comparative Example 72 at room temperature in FIG. 3 a) and at 55° C. in FIG. 3 b).

FIG. 4 shows the yields of difluoromethyldiazomethane as per Examples 73 to 79.

FIG. 1 shows that reacting 2,2-difluoroethylamine 1 with tert-butyl nitrite by use of acetic acid (AcOH) gives 2,2-difluoromethyldiazomethane 2. In accordance with the present disclosure, the 2,2-difluoromethyldiazomethane 2 is prepared in a continuous process in a reactor 5. In the illustrated embodiment, difluoroethylamine 1 is initially charged to a first vessel, while tert-butyl nitrite and acetic acid are initially charged conjointly to a second vessel. Before entry into the reactor 5, 2,2-difluoroethylamine 1, tert-butyl nitrite and acetic acid are mixed in the mixer 4.

The mixer 4 is embodied in FIG. 1 as a T-piece. 2,2-Difluoroethylamine 1, tert-butyl nitrite and acetic acid are each fed to the mixer 4 by a pump 3. A back pressure valve 6 is connected to the reactor 5.

FIG. 2 shows the conversion of a β,β-difluoroalkylamine as per formula (1) 7 with tert-butyl nitrite by use of acetic acid (AcOH) into a 2,2-difluorinated diazoalkane 8. In accordance with the present disclosure, the fluorinated diazoalkane 8 is prepared in a continuous process in a reactor 5. In the illustrated embodiment, the β,β-difluoroalkylamine 7 is initially charged to a first vessel, while tert-butyl nitrite and acetic acid are initially charged conjointly to a second vessel. The β,β-difluoroalkylamine 7, tert-butyl nitrite and acetic acid are fed by a pump 3 to a T-piece mixer 4, and mixed before passing into the reactor 5. A back pressure valve 6 is connected to the reactor 5.

The fluorinated diazoalkane 8 is fed to a second T-piece mixer 4. It is likewise supplied with a dipolarophile D by a further pump 3. The fluorinated diazoalkane 8 and the dipolarophile D are mixed in the second mixer 4 and directed into a second reactor 5. A further back pressure valve 6 is connected also to the second reactor 5. The fluorinated diazoalkane 8 and the dipolarophile D undergo a 1,3-dipolar cycloaddition reaction in the second reactor 5 to form a fluoroalkyl-substituted compound.

### Material:

Chloroform was dried over calcium hydride before use.

### EXAMPLE 1

### Continuous Preparation of Difluoromethyldiazomethane

A solution of difluoroethylamine (0.2 M, 4 eq) in chloroform was initially charged to a syringe. A further syringe was initially charged with a mixture of 40 mol % acetic acid (1.6 eq) and tert-butyl nitrite (0.24 M, 4.8 eq) in chloroform at room temperature (20±2° C.). Both solutions were syringe pumped (Chemyx Fusion ≤V710) at a constant flow rate of 50 μL/min into a micromixer (Little Things Factory, MR-LAB Type MST, volume 200 μL). This micromixer also served as microreactor. The reactor had connected to it a back pressure valve (IDEX back pressure assembly) which established a pressure of 40 psi. The micromixer/microreactor was heated to a temperature of 75° C. The mixture of the reactants had a residence time of 2 minutes in the reactor.

A sample of the reaction solution was taken at the outlet of the reactor and analysed by NMR. Difluoromethyldiazomethane was obtained in a yield of about 40%.

The reaction of difluoroethylamine (4 eq) with tert-butyl nitrite (4.8 eq) was repeated using various proportions of acetic acid, temperatures and residence times in the reactor. Samples of the reaction solution were in each case taken at the output of the reactor and analysed by NMR. The conditions and yields of the individual reactions are summarised below in Table 1.

TABLE 1
Continuous conversion of difluoroethylamine
with (1.2 eq) tert-butyl nitrite into
difluoromethyldiazo-methane

| | Reaction conditions | | | |
|---|---|---|---|---|
| | | | Residence | |
| | Acetic | Temper- | time in | |
| | acid, | ature, | reactor, | Yield, |
| Example | mol % | ° C. | min | % |
| 2 | 20 mol % | 75° C. | 2 min | 30% |
| 3 | 10 mol % | 55° C. | 2 min | 11% |
| 4 | 10 mol % | 75° C. | 8 min | 15% |
| 5 | 40 mol % | 90° C. | 2 min | 27% |
| 6 | 10 mol % | 75° C. | 2 min | 29% |
| 7 | 10 mol % | 75° C. | 4 min | 23% |
| 8* | 5 mol % | 55° C. | 10 min | 26% |

*a 0.8 mL capacity microreactor was additionally attached to the micromixer. The combined capacity of micromixer and microreactor was 1 mL.

It transpired that a temperature of 55° C. to 90° C., a 2 to 10 minutes' residence time for the components in the mixer/reactor and an acetic acid amount-of-substance fraction ranging from 10 mol % to 40 mol % gave good yields of difluoromethyldiazomethane.

### EXAMPLE 9

### Use of Said Difluoromethyldiazomethane in Cyclopropanations

Difluoromethyldiazomethane was prepared as described in Example 1. The reaction solution from the reactor was passed under argon into a 50 ml reaction flask containing 0.4 mmol of styrene (1 eq) and, by way of catalyst, 5 mol % of rhodium(II) (Rh₂esp₂) in 4 mL of CHCl₃ and stirred at room temperature (20±2° C.) for 4 hours. Subsequently, the chloroform was removed under reduced pressure and the residue was purified by column chromatography. 1-Phenyl-2-difluoromethylcyclopropane was obtained in a yield of 67%.

The conversion of the reaction solution from the reactor was repeated using various styrene derivatives. The styrene derivatives used, the difluoromethyl-substituted cyclopropanes obtained and the respective yields are summarised below in Table 2.

TABLE 2
Conversion of difluoromethyldiazomethane with
styrene derivatives into difluoromethyl-substituted
cyclopropanes

| Ex- | | | Yield, |
|---|---|---|---|
| ample | Styrene reactant | Cyclopropane | % |
| 10 | 4-methylstyrene | 1-(4-methylphenyl)-2- | 68 |
| | | difluoromethylcyclopropane | |
| 11 | 4-tert- | 1-(4-tert-butylphenyl)-2- | 57 |
| | butylstyrene | difluoromethylcyclopropane | |
| 12 | 4-fluorostyrene | 1-(4-fluorophenyl)-2- | 42 |
| | | difluoromethylcyclopropane | |
| 13 | 4-chlorostyrene | 1-(4-chlorophenyl)-2- | 56 |
| | | difluoromethylcyclopropane | |
| 14 | 4-bromostyrene | 1-(4-bromophenyl)-2- | 59 |
| | | difluoromethylcyclopropane | |
| 15 | 3-methylstyrene | 1-(3-methylphenyl)-2- | 60 |
| | | difluoromethylcyclopropane | |
| 16 | 3-fluorostyrene | 1-(3-fluorophenyl)-2- | 52 |
| | | difluoromethylcyclopropane | |
| 17 | 3-chlorostyrene | 1-(3-chlorophenyl)-2- | 34 |
| | | difluoromethylcyclopropane | |
| 18 | 2-fluorostyrene | 1-(2-fluorophenyl)-2- | 49 |
| | | difluoromethylcyclopropane | |
| 19 | 2-vinylnaphthalene | 1-(2-naphthalenyl)-2- | 32 |
| | | difluoromethylcyclopropane | |
| 20 | alpha- | (2-(difluoromethyl)-1- | 63 |
| | methylstyrene | methylcyclopropyl)benzene | |
| 21 | trans-beta- | (2-(difluoromethyl)-3- | 45 |
| | methylstyrene | methylcyclopropyl)benzene | |
| 22 | indene | 1-(difluoromethyl)- | 50 |
| | | 1,1a,6,6a-tetrahydrocyclo- | |
| | | propa[a]indane | |
| 23 | dihydro- | 1-(difluoromethyl)- | 49 |
| | naphthalene | 1a,2,3,7b-tetrahydro-1H- | |
| | | cyclopropa[a]naphthalene | |
| 24 | 4-vinylpyridine | 1-(4-pyridyl)-2- | <10 |
| | | difluormethyl-cyclopropane | |

As is derivable from Table 2, the process makes for an efficient synthesis of difluoromethyl-substituted cyclopropanes in good yield. In an embodiment, one requirement for this may be the continuous preparation of the difluoromethyldiazomethane in good yield in the first step. As is further derivable from Table 2, the use of heterocycles resulted in but low yields of the desired cyclopropane.

### EXAMPLE 25

### Preparation of Methyl 3-(Difluoromethyl)-1H-Pyrazole-5-Carboxylate

### a) Preparing the Fluorinated Diazoalkane

Two stock solutions were prepared. 2,2-Difluoroethylamine (0.1 M, 2 eq) was dissolved in 9 mL of CHCl₃. tBuONO (0.24 M, 2.4 eq) and 5 mol % of acetic acid (0.08 M, 0.1 eq) were likewise dissolved in 9 mL of CHCl₃. Both solutions were initially charged to a syringe and added by syringe pumping at a flow rate of 50 μL/min via a micromixer (Little Things Factory, MR Lab Type MST) having a capacity of 0.2 mL and a microreactor (CS Chromatographie PTFE tubing, Article No. 590515, ID=0.8 mm, length 1.6 m) having a volume of 0.8 mL. The micromixer and the microreactor were heated to 55° C. The mixture of the reactants had a residence time of 10 minutes in the microreactor.

b) Reacting the Fluorinated Diazoalkane with a Dipolarophile in a 1,3-Dipolar Cycloaddition Reaction

The downstream tube end of the reactor was directed into a flask which was initially charged with 1 eq of ethyl propiolate (0.5 mmol) dissolved in 6 mL of chloroform. On completion of the addition the reaction solution was stirred overnight at room temperature (20±2° C.). The solvent was removed under reduced pressure and the residue was purified by column chromatography to obtain methyl 3-(difluoromethyl)-1H-pyrazole-5-carboxylate.

The reaction was repeated with various dipolarophiles. The di- and trifluoromethyl-substituted pyrazoles obtained, the dipolarophiles and the particular fluoroalkyl groups are summarised below in Table 3.

TABLE 3
Preparation of various di- and trifluoromethyl-
substituted pyrazoles and pyrazolines

| Ex- | Di- or trifluoromethyl- | | Fluoroalkyl |
|---|---|---|---|
| ample | substituted pyrazole | Dipolarophile | group |
| 26 | ethyl 3-(difluoromethyl)-1H- | propiolate | CF₂H |
| | pyrazole-5-carboxylate | | |
| 27 | tert-butyl | propiolate | CF₂H |
| | 3-(difluoromethyl)-1H- | | |
| | pyrazole-5-carboxylate | | |
| 28 | 5-benzyl-3-(difluoromethyl)- | Maleimide | CF₂H |
| | 1,6a-dihydropyrrolo[3,4- | | |
| | c]pyrazole-4,6(3aH,5H)-dione | | |
| 29 | 1-(3-(difluoromethyl)-1H- | alkynyl | CF₂H |
| | pyrazol-5-yl)ethanone | ketone | |
| 30 | dimethyl 3-(difluoromethyl)- | alkynyl | CF₂H |
| | 1H-pyrazole-4,5- | diester | |
| | dicarboxylate | | |
| 31 | tert-butyl | acrylic ester | CF₂H |
| | 3-(difluoromethyl)-4,5- | | |
| | dihydro-1H-pyrazole-5- | | |
| | carboxylate | | |
| 32 | 1-(3-(difluoromethyl)-4,5- | vinyl ketone | CF₂H |
| | dihydro-1H-pyrazol-5- | | |
| | yl)ethanone | | |
| 33 | tert-butyl | propiolate | CF₃ |
| | 3-(trifluoromethyl)-1H- | | |
| | pyrazole-5-carboxylate | | |
| 34 | methyl 3-(trifluoromethyl)- | propiolate | CF₃ |
| | 1H-pyrazole-5-carboxylate | | |
| 35 | ethyl 3-(trifluoromethyl)- | propiolate | CF₃ |
| | 1H-pyrazole-5-carboxylate | | |
| 36 | 1-(3-(trifluoromethyl)-1H- | alkynyl | CF₃ |
| | pyrazol-5-yl)ethanone | ketone | |
| 37 | dimethyl | alkynyl | CF₃ |
| | 3-(trifluoromethyl)-1H- | diester | |
| | pyrazole-4,5-dicarboxylate | | |
| 38 | 1-(3-(trifluoromethyl)-4,5- | vinyl ketone | CF₃ |
| | dihydro-1H-pyrazol-5- | | |
| | yl)ethanone | | |
| 39 | tert-butyl | acrylic ester | CF₃ |
| | 3-(trifluoromethyl)-4,5- | | |
| | dihydro-1H-pyrazole-5- | | |
| | carboxylate | | |

As is derivable from Table 3, the process makes for an efficient synthesis of fluoroalkyl-substituted pyrazoles and pyrazolines. In an embodiment, one requirement for this may be the continuous preparation of the fluorinated diazo compound in good yield in the first step.

### EXAMPLE 40

### Preparation of 3-(Phenylsulfonyl)-5-(Difluoromethyl)-4,5-Dihydro-1H-Pyrazole

### a) Preparing the Fluorinated Diazoalkane

Two stock solutions were prepared. 2,2-Difluoroethylamine (0.2 M, 4 eq) was dissolved in CHCl₃. tert-Butyl nitrite (0.24 M, 4.8 eq) and 5 mol % of acetic acid (0.01 M, 0.2 eq) were likewise dissolved in CHCl₃. Both solutions were initially charged to a syringe and added by syringe pumping at a flow rate of 50 μL/min via a micromixer (Little Things Factory, MR Lab Type MST) having a capacity of 0.2 mL and a microreactor (CS Chromatographie PTFE tubing, Article No. 590515, ID=0.8 mm, length 1.6 m) having a volume of 0.8 mL. The micromixer and the microreactor were heated to 55° C. The mixture of the reactants had a residence time of 10 minutes in the microreactor.

b) Reacting the Fluorinated Diazoalkane with a Dipolarophile in a 1,3-Dipolar Cycloaddition Reaction

The downstream tube end of the reactor was directed into a flask which was initially charged with 1 eq of phenyl vinyl sulfone (0.5 mmol) dissolved in chloroform. On completion of the addition the reaction solution was stirred for 24 hours at room temperature (20±2° C.). The solvent was removed under reduced pressure and the residue was purified by column chromatography to obtain 3-(phenylsulfonyl)-5-(difluoromethyl)-4,5-dihydro-1H-pyrazole.

The reaction was repeated with various vinyl sulfones. The di- and trifluoromethyl-substituted pyrazoles obtained, the vinyl sulfones used and the particular fluoroalkyl groups are summarised below in Table 4.

TABLE 4
Preparation of various di- and
trifluoromethylsulfonyl-substituted pyrazoles and
pyrazolines

| | Di- or | | Fluoro- |
|---|---|---|---|
| Ex- | trifluoromethylsulfonyl- | Dipolaro- | alkyl |
| ample | substituted pyrazole | phile | group |
| 41 | 3-(methylsulfonyl)-5- | vinyl | CF₃ |
| | (trifluoromethyl)-4,5-dihydro- | sulfone | |
| | 1H-pyrazole | | |
| 42 | 3-(ethylsulfonyl)-5- | vinyl | CF₃ |
| | (trifluoromethyl)-4,5-dihydro- | sulfone | |
| | 1H-pyrazole | | |
| 43 | 3-(benzylsulfonyl)-5- | vinyl | CF₃ |
| | (trifluoromethyl)-4,5-dihydro- | sulfone | |
| | 1H-pyrazole | | |
| 44 | 3-(phenylsulfonyl)-5- | vinyl | CF₃ |
| | (trifluoromethyl)-4,5-dihydro- | sulfone | |
| | 1H-pyrazole | | |
| 45 | 3-((4-methylphenyl)sulfonyl)- | vinyl | CF₃ |
| | 5-(trifluoromethyl)-4,5- | sulfone | |
| | dihydro-1H-pyrazole | | |
| 46 | 3-((4-fluorophenyl)sulfonyl)- | vinyl | CF₃ |
| | 5-(trifluoromethyl)-4,5- | sulfone | |
| | dihydro-1H-pyrazole | | |
| 47 | 3-((4-chlorophenyl)sulfonyl)- | vinyl | CF₃ |
| | 5-(trifluoromethyl)-4,5- | sulfone | |
| | dihydro-1H-pyrazole | | |
| 48 | 3-((4-bromophenyl)sulfonyl)-5- | vinyl | CF₃ |
| | (trifluoromethyl)-4,5-dihydro- | sulfone | |
| | 1H-pyrazole | | |
| 49 | 3-((4-methoxyphenyl)sulfonyl)- | vinyl | CF₃ |
| | 5-(trifluoromethyl)-4,5- | sulfone | |
| | dihydro-1H-pyrazole | | |
| 50 | 3-((3-methylphenyl)sulfonyl)- | vinyl | CF₃ |
| | 5-(trifluoromethyl)-4,5- | sulfone | |
| | dihydro-1H-pyrazole | | |
| 51 | 3-((2-methylphenyl)sulfonyl)- | vinyl | CF₃ |
| | 5-(trifluoromethyl)-4,5- | sulfone | |
| | dihydro-1H-pyrazole | | |
| 52 | 3-(methylsulfonyl)-5- | vinyl | CF₂H |
| | (difluoromethyl)-4,5-dihydro- | sulfone | |
| | 1H-pyrazole | | |
| 53 | 3-(ethylsulfonyl)-5- | vinyl | CF₂H |
| | (difluoromethyl)-4,5-dihydro- | sulfone | |
| | 1H-pyrazole | | |
| 54 | 3-(benzylsulfonyl)-5- | vinyl | CF₂H |
| | (difluoromethyl)-4,5-dihydro- | sulfone | |
| | 1H-pyrazole | | |
| 55 | 3-(phenylsulfonyl)-5- | vinyl | CF₂H |
| | (difluoromethyl)-4,5-dihydro- | sulfone | |
| | 1H-pyrazole | | |
| 56 | 3-((4-methylphenyl)sulfonyl)- | vinyl | CF₂H |
| | 5-(difluoromethyl)-4,5- | sulfone | |
| | dihydro-1H-pyrazole | | |
| 57 | 3-((4-fluorophenyl)sulfonyl)- | vinyl | CF₂H |
| | 5-(difluoromethyl)-4,5- | sulfone | |
| | dihydro-1H-pyrazole | | |
| 58 | 3-((4-chlorophenyl)sulfonyl)- | vinyl | CF₂H |
| | 5-(difluoromethyl)-4,5- | sulfone | |
| | dihydro-1H-pyrazole | | |
| 59 | 3-((4-bromophenyl)sulfonyl)-5- | vinyl | CF₂H |
| | (difluoromethyl)-4,5-dihydro- | sulfone | |
| | 1H-pyrazole | | |
| 60 | 3-((4-methoxyphenyl)sulfonyl)- | vinyl | CF₂H |
| | 5-(difluoromethyl)-4,5- | sulfone | |
| | dihydro-1H-pyrazole | | |
| 61 | 3-((3-methylphenyl)sulfonyl)- | vinyl | CF₂H |
| | 5-(difluoromethyl)-4,5- | sulfone | |
| | dihydro-1H-pyrazole | | |
| 62 | 3-((2-methylphenyl)sulfonyl)- | vinyl | CF₂H |
| | 5-(difluoromethyl)-4,5- | sulfone | |
| | dihydro-1H-pyrazole | | |
| 63 | 3-(methylsulfonyl)-5-(methyl)- | vinyl | branched |
| | 5-(trifluoromethyl)-4,5- | sulfone | alkyl |
| | dihydro-1H-pyrazole | | |
| 64 | 3-(phenylsulfonyl)-5-(methyl)- | vinyl | branched |
| | 5-(trifluoromethyl)-4,5- | sulfone | alkyl |
| | dihydro-1H-pyrazole | | |
| 65 | 3-(methylsulfonyl)-5- | vinyl | branched |
| | phenethyl-5-(trifluoromethyl)- | sulfone | alkyl |
| | 4,5-dihydro-1H-pyrazole | | |
| 66 | 3-(methylsulfonyl)-5-(phenyl)- | vinyl | branched |
| | 5-(trifluoromethyl)-4,5- | sulfone | alkyl |
| | dihydro-1H-pyrazole | | |
| 67 | 3-(methylsulfonyl)-5-(4- | vinyl | branched |
| | methylphenyl)-5- | sulfone | alkyl |
| | (trifluoromethyl)-4,5-dihydro- | | |
| | 1H-pyrazole | | |
| 68 | 3-(methylsulfonyl)-5-(4- | vinyl | branched |
| | fluorophenyl)-5- | sulfone | alkyl |
| | (trifluoromethyl)-4,5-dihydro- | | |
| | 1H-pyrazole | | |

As is derivable from Table 4, the process makes for an efficient synthesis of fluoroalkyl-substituted and sulfonyl-substituted pyrazoles and pyrazolines. In an embodiment, one requirement for this may be the continuous preparation of the fluorinated diazo compound in good yield in the first step.

### EXAMPLE 69

### Preparation of 3-(Phenylsulfonyl)-5-(Difluoromethyl)-4,5-Dihydro-1H-Pyrazole

### a) Preparing the Fluorinated Diazoalkane

Two stock solutions were prepared. 2,2-Difluoroethylamine (0.4 M, 4 eq) was dissolved in CHCl₃. tert-Butyl nitrite (0.48 M, 4.8 eq) and 5 mol % of acetic acid (0.16 M, 0.2 eq) were likewise dissolved in CHCl₃. Both solutions were initially charged to a syringe and added by syringe pumping at a flow rate of 50 μL/min via a micromixer (Little Things Factory, MR Lab Type MST) having a capacity of 0.2 mL and a microreactor (CS Chromatographie PTFE tubing, Article No. 590515, ID=0.8 mm, length 1.6 m) having a volume of 0.8 mL. The micromixer and the microreactor were heated to 55° C. The mixture of the reactants had a residence time of 10 minutes in the microreactor.

b) Reacting the Fluorinated Diazoalkane with a Dipolarophile in a 1,3-Dipolar Cycloaddition Reaction in a Second Microreactor

A third stock solution was prepared: phenyl vinyl sulfone (0.1 M, 1 eq) was dissolved in CHCl₃. This solution was initially charged to a syringe and added by syringe pumping at a flow rate of 50 μL/min together with the downstream tubing end of the first reactor connected to a second micromixer (Little Things Factory, MR Lab Type MST) and a microreactor (CS Chromatographie PTFE tubing, Article No. 590515, ID=0.8 mm, length 2.6 m) having a capacity of 1.3 mL. The micromixer and the microreactor were heated to 55° C. The downstream tubing end of the second reactor was connected to a back pressure valve (20 psi) and then directed into a round bottom flask. On completion of the addition of the reactants into the micromixers/microreactors, the collected reacted solution was desolventised under reduced pressure and the residue was purified by column chromatography to obtain 3-(phenylsulfonyl)-5-(difluoromethyl)-4,5-dihydro-1H-pyrazole.

### EXAMPLE 70

### Gramwise Preparation of 3-(Phenylsulfonyl)-5-(Difluoromethyl)-4,5-Dihydro-1H-Pyrazole

### a) Preparing the Fluorinated Diazoalkane

Two stock solutions were prepared. 2,2-Difluoroethylamine (0.4 M, 4 eq) was dissolved in CHCl₃. Tert-Butyl nitrite (0.48 M, 4.8 eq) and 5 mol % acetic acid (0.02 M, 0.2 eq) were likewise dissolved in CHCl₃. Altogether 50 mL of both stock solutions (corresponding to 20 mmol of 2,2-difluoroethylamine) were prepared. Compared with the preparation of 3-(phenylsulfonyl)-5-(difluoromethyl)-4,5-dihydro-1H-pyrazole as per Example 40, the amine solution had twice the concentration. Both solutions were initially charged at room temperature (20±2° C.) to a syringe and added by syringe pumping at a flow rate of 50 μL/min via a micromixer (Little Things Factory, MR Lab Type MST) having a capacity of 0.2 mL and a microreactor (CS Chromatographie PTFE tubing, Article No. 590515, ID=0.8 mm, length 1.6 m) having a volume of 0.8 mL. The micromixer and the microreactor were heated to 55° C. The mixture of the reactants had a residence time of 10 minutes in the microreactor.

At a flow rate of 50 μL/min, the addition time for the 50 mL volumes used in both cases was altogether 16.6 hours.

b) Reacting the Fluorinated Diazoalkane with a Dipolarophile in a 1,3-Dipolar Cycloaddition Reaction

The downstream tube end of the reactor was directed into a flask which was initially charged with 1 eq of phenyl vinyl sulfone (5 mmol) dissolved in chloroform. On completion of the addition the reaction solution was stirred for a further 24 hours at room temperature (20±2° C.). The solvent was removed under reduced pressure and the residue was purified by column chromatography to obtain 1.08 g of 3-(phenylsulfonyl)-5-(difluoromethyl)-4,5-dihydro-1H-pyrazole.

The amount of product obtained shows that fluorinated diazoalkane was made in gram amounts for the synthesis of step b). The gramwise preparation of 3-(phenylsulfonyl)-5-(difluoromethyl)-4,5-dihydro-1H-pyrazole required an addition time of about 17 hours. Yet the continuously prepared fluorinated diazoalkane did not accumulate, ensuring process safety even at the high concentrations, volumes and reaction times used.

### COMPARATIVE EXAMPLE 71

### Preparation of 2,2-Difluoromethyldiazomethane by Conjoint Initial Charging of Reactants

In a comparative experiment, difluoroethylamine and tert-butyl nitrite were initially charged conjointly at room temperature. Difluoroethylamine (1 eq) in chloroform was initially charged to a 50 mL flask. 5 mol % of acetic acid and tert-butyl nitrite (1.2 eq) were added and the reaction mixture was stirred at room temperature (20±2° C.). Samples of the reaction solution were taken at regular intervals and analysed by NMR. Table 5 below contains data points from this experiment and indicates the amount-of-substance fraction of amine and of diazo compound in the initially charged solution.

TABLE 5
Amine and diazo compound amount-of-
substance fractions on conjoint initial
charging of starting materials

| | | Diazo |
|---|---|---|
| Time | Amine | compound |
| 30 min | 93% | 2% |
| 120 min | 72% | 4% |
| 180 min | 26% | 3% |

As is derivable from Table 5, within just three hours, the amount of substance decreased significantly for the difluoroethylamine reactant. The desired diazo compound was only formed to a minimal extent. It is believed that a background reaction led to premature consumption of the amine and hence to a reduction in the yield of diazo compound. This experiment shows that the conjoint initial charging of all three starting materials—amine, nitrite and acetic acid—to one vessel at room temperature is unsuitable for above 2 hour addition times into the microreactor.

### COMPARATIVE EXAMPLE 72

### Preparation of 2,2-Difluoromethyldiazomethane by Conjoint Initial Charging of Starting Materials

In further comparative experiments in the batch mode, 162 mg (2 mmol) of 2,2-difluoroethylamine, 2.4 mmol of tert-butyl nitrite (1.2 eq) and 0.2 mmol of acetic acid (5 mol %) in 2 ml of CDCl₃ were in each case initially charged conjointly. α,α,α-Trifluorotoluene was added as internal standard and the reaction vessels were sealed. A comparative batch was stirred at room temperature (20±2° C.) and samples were taken after 30, 60, 120 and 180 minutes. Further comparative batches were stirred at 55° C. for 5, 15, 30, 60, 120 and 180 minutes and, after the particular reaction time, briefly cooled in an ice bath before a sample was taken and analysed by NMR.

FIGS. 3a) and 3 b) show the respective yields of difluoromethyldiazomethane and the residual amounts of difluoroethylamine for the experiments at 55° C. and at room temperature. As is derivable from FIG. 3 a), the amount of the difluoroethylamine decreased rapidly at 55° C. while merely small amounts of difluoromethyldiazomethane were formed. The highest yield of 15% was obtained after a reaction time of 15 minutes. At room temperature, merely traces of difluoromethyldiazomethane were detected, while the starting amount of difluorethylamine likewise decreased to about 70% after 2 hours.

This shows that on conjoint initial charging of the amine, nitrite and acetic acid starting materials to one vessel at 55° C., the maximum yield of difluoromethyl diazomethane was 15% after 15 minutes. If all starting materials were charged together at room temperature, only traces of difluoromethyl diazomethane were formed. Both at room temperature and at 55° C., the reactants decompose under these conditions.

This illustrates that neither at elevated temperatures nor at room temperature sufficiently high yields for an industrial production can be achieved if the starting materials β,β-difluoroalkylamine and nitrite are initially charged together in a vessel. A continuous process, on the other hand, makes it possible to achieve consistently high yields over a prolonged period of time, which, for example, enable further chemical transformations.

### EXAMPLES 73-79

### Preparation of Difluoromethyldiazomethane by Continuous Flow

0.2 M of 2,2-difluoroethylamine was dissolved in anhydrous devolatilised CHCl₃. In a further vessel, 0.24 M of tert-butyl nitrite and 0.08 M of acetic acid were dissolved in anhydrous devolatilised CHCl₃. Both solutions were filled into syringes, introduced into a syringe pump (Chemyx Fusion V710) and connected via PTFE tubing to a micromixer having an internal volume of 200 μL (Little Things Factory, MR-LAB Type MST). The micromixer likewise served as microreactor, a back pressure valve (IDEX back pressure assembly) creating a pressure of 20 psi. At the outlet, the particular reaction mixture was removed and analysed as a whole by NMR.

Samples using 10, 20 or 40 mol % of acetic acid were transported at respectively constant flow rates of 13, 25 or 50 μL/min into the reactor, which was preheated to temperatures of 55° C., 75° C. and 90° C. The particular examples are summarised below in Table 6.

TABLE 6
Examples 73-79

| Ex- | Flow rate | Temperature | Acetic acid |
|---|---|---|---|
| ample | [μL/min] | [° C.] | [mol %] |
| 73 | 50 | 55 | 10 |
| 74 | 50 | 75 | 10 |
| 75 | 25 | 75 | 10 |
| 76 | 13 | 75 | 10 |
| 77 | 50 | 75 | 20 |
| 78 | 50 | 75 | 40 |
| 79 | 50 | 90 | 40 |

FIG. 4 shows the respectively obtained yields of difluoromethyldiazomethane reported in % on the Y-axis. As is derivable from FIG. 4, in contradistinction to the batch reaction, a yield of more than 10% was obtained continuously in all experiments. The reaction mixture may further be added directly to subsequent reactions. Scalability is obtainable by adding the volume flow across a longer period, or by parallelising the microreactors.

1. A process for preparing a fluorinated diazoalkane wherein the process is a continuous process and a β,β-difluoroalkylamine is reacted with an organic nitrite in a reactor, wherein the β,β-difluoroalkylamine and the organic nitrite are initially charged to separate vessels.

2. The process according to claim 1, wherein the β,β-difluoroalkylamine is initially charged to a first vessel and the organic nitrite and acetic acid are initially charged conjointly to a second vessel, or the organic nitrite is initially charged to a first vessel and the β,β-difluoroalkylamine and acetic acid are initially charged conjointly to a second vessel.

3. The process according to claim 1, wherein the reaction of the β,β-difluoroalkylamine with the organic nitrite in the reactor is carried out at a temperature in the range of approximately ≥40° C. to ≤100° C., preferably in the range of approximately ≥40° C. to ≤80° C., preferably at a temperature in the range of approximately ≥55° C. to ≤75° C.

4. The process according to claim 2, wherein the residence time of the components β,β-difluoroalkylamine, organic nitrite and acetic acid in the reactor is in a range of approximately ≥30 seconds to ≤1 hour, preferably in the range of approximately ≥1 min to ≤20 min and preferably in the range of approximately ≥2 min to ≤10 min.

5. The process according to claim 1, wherein the organic nitrite is used in a ≥0.5-fold to ≤2-fold, preferably a ≥1-fold to ≤1.5-fold and more preferably a ≥1-fold to ≤1.2-fold molar excess based on the β,β-difluoroalkylamine.

6. The process according to claim 2, wherein the amount-of-substance fraction of acetic acid is in the range of approximately ≥1 mol % to ≤50 mol %, preferably in the range of approximately ≥5 mol % to ≤40 mol % and more preferably in the range of approximately ≥5 mol % to ≤10 mol %, based on the amount of substance of the β,β-difluoroalkylamine.

7. The process according to claim 1, wherein the β,β-difluoroalkylamine has the following general formula (1) 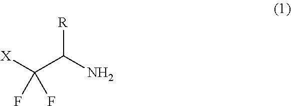
where:
R is selected from the group comprising H and/or substituted or unsubstituted alkyl, aryl, arylalkyl, cyclyl, heteroaryl or heterocyclyl,
X is selected from the group comprising H, F, Cl, CN, CO₂R′, CONR′, COR′, SO₂R′, SO₂NR′₂ and/or substituted or unsubstituted alkyl, aryl, arylalkyl, cyclyl, heteroaryl or heterocyclyl, and
R′ is selected, independently where applicable, from the group comprising H and/or substituted or unsubstituted alkyl, aryl, arylalkyl, cyclyl, heteroaryl or heterocyclyl.

8. Use of the process according to any one of the preceding claims for preparing a fluoroalkyl-substituted compound, especially a di- or trifluoroalkyl-substituted pyrazole, pyrazoline or cyclopropane.

9. A process for preparing a fluoroalkyl-substituted compound, especially a di- or trifluoroalkyl-substituted pyrazole, pyrazoline or cyclopropane, wherein the process comprises the steps of:
a) preparing a fluorinated diazoalkane as per the process according to any one of claims 1-7, and
b) reacting the fluorinated diazoalkane in a cyclopropanation reaction or with a dipolarophile in a 1,3-dipolar cycloaddition reaction.

10. The process according to claim 9, wherein the dipolarophile has the following general formula (2) or (3) 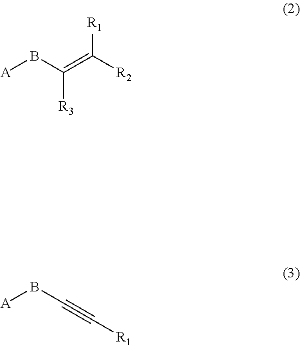
where:
A is selected from the group comprising substituted or unsubstituted alkyl, aryl, arylalkyl, cyclyl, heteroaryl, heterocyclyl, alkoxy, aryloxy, NR″₂ and/or NHR″,
B is selected from the group comprising CO and/or SO₂,
R₁, R₂ and R₃ are each independently selected from the group comprising H, F, Cl, C₁₋₃-alkyl, CF₃, CF₂H, CFH₂, phenyl, COR′″ and/or SO₂R′″,
R″ is selected, independently where applicable, from the group comprising C₁₋₈-alkyl, phenyl and/or C₅₋₆-heteroaryl, and
R′″ is selected from the group comprising H and/or substituted or unsubstituted alkyl, aryl, arylalkyl, cyclyl, heteroaryl or heterocyclyl,
where in the case of the dipolarophile of formula (2) A and R₁ are capable of conjointly forming a 5-, 6- or 7-membered ring which is present as ketone, lactone, lactam, sulfone, sulfonic ester, sultam, anhydride or imide.
